(19) United States
(12) Patent Application Publication
(10) Pub. No.: US 2004/0076647 A1
(43) Pub. Date: Apr. 22, 2004

(54) Liquid collagen hemostat

(76) Inventor: Wolfgang Biering, Wien (AT)

Correspondence Address: OSTROLENK FABER GERB & SOFFEN, 1180 AVENUE OF THE AMERICAS, NEW YORK, NY, 100368403

(21) Appl. No.: 10/312,157

(22) PCT Filed: Jun. 15, 2001
(86) PCT No.: PCT/EP01/06793

(30) Foreign Application Priority Data
Jun. 20, 2000 (AT) A 1070/2000

(51) Int. Cl. A61K 9/00

(52) U.S. Cl. 424/400

(57) ABSTRACT

Subject of the invention is a collagen hemostat based on liquid collagen of neutral pH and of low density. Its specific weight is in the range of 0.07 to 0.9 mg/cm³. The hemostat according to the invention preferably contains additives such as foaming additives or physiologically active substances. The further subject of the invention is the method of producing the collagen hemostat by acidifying a source of native collagen, heating the collagen to a temperature of 30-50° C. under agitation, neutralizing the collagen by addition of an alkaline solution and expanding the collagen to obtain a liquid of low density. The collagen hemostat according to the invention can be used in various surgical interventions, in particular of laparoscopic procedures.

The invention relates to a liquid collagen hemostat based on expanded collagen, a method of preparing the hemostat and its use in surgical interventions.

Collagen hemostats have been successfully used in the management of a variety of wounds. Collagen is an adherence protein to which blood platelets attach, when they escape from the vascular system caused by an injury of blood vessels. The vessels comprising the vascular system are surrounded by networks of collagen fibrils which are also present throughout the tissues of the body. Upon platelet attachment to collagen the binding of further platelets is initiated resulting in platelet aggregation and activation, which is the main principle of primary hemostasis to stop bleeding at the site of injury.

Microfibrillar collagen is used extensively for wide-area parenchyma bleeding and for laparoscopic procedures in which a specific amount of hemostatic agent can be delivered. On the other hand hemostatic agents in the form of collagen sponges have been shown to be particularly attractive in dermatological applications where adherence to the wound site and ease of removal are important considerations.

Despite these differences in delivery and handling, the primary function of these hemostatic agents is the rapid initiation of hemostatic plug formation through platelet adhesion, platelet activation, and gross blood coagulation.

The choice of the collagen hemostatic formulation, such as sponges, particles or fluids, mainly depends on the indication to treat either large surfaces or punctual sites of injuries. It is well known in the art to use the collagen in its fibrillar form. Collagen fibrils are solid and easily dispersed in an aqueous medium. It is dissolved at an acid pH and solid fibrils are precipitated when neutralized, to obtain a highly viscous gel and the fibrils immersed therein.

Collagen in its fibrillar form is produced according to WO 9806444. First fetal and adult collagen is dissolved to get a solution of pH 4.0 to 6.0. This composition is suitable for packaging in an aerosol container for dispensing as a blood coagulation foam. Upon neutralization the collagen is precipitated as fibrils and thus gelling and clotted either by the coincident spraying of a neutralizing solution, or by the production of pellets resulting from centrifugation of a neutralized solution of collagen.

A dispersion of solid collagen that is mixed with thrombin is produced according to WO 9831403 by first drying the mixture and reconstituting the solid to a biomaterial that contains the solid as small particles which are flowable through a cannula of less than 1 mm. Thrombin is the activated form of the blood coagulation factor prothrombin and has enzymatic action on fibrinogen to form fibrin and thus promotes the formation of a blood clot.

A collagen gel is further described in the WO 9952946, that is obtained as a neutralized solution by specific temperature control. Thus, fibrils are not precipitated in the neutral state and the collagen can be sterilized and presented as a liquid. A similar gel is the basis for the production of hemostatic sponges according to WO 9737694. Upon lyophilization of the gel a sponge-like fleece is produced.

The above mentioned materials based on solid collagen and fibrils might not be useful for the surgical application where a quick hemostasis is necessary, because first the solid has to get intimate contact with blood to initiate hemostasis. Likewise it is difficult to apply a collagen liquid to a wound surface because of its high viscosity.

It is the object of the invention to provide a hemostatic material based on liquid collagen that is easy to apply during surgery and immediately developing hemostatic action when contacted with blood. It should be specifically useful for laparoscopic procedures.

The object is solved by the subject matter of the invention represented by a collagen hemostat according to the present claims. This hemostat is based on liquid collagen with a pH in the range of 6-8, preferably between 6.5 and 8.0, and characterized by a specific weight of 0.07-0.9 mg/cm³, a preferred range is 0.1-0.7 mg/cm³, that is obtained after expansion of the liquid. During expansion of a collagen liquid having a concentration of about 1-40 mg/ml, preferably between 1-20 mg/ml, or even higher concentrations up to 50 mg/ml the liquid is increasing its surface and contact surface with the surrounding gas, e.g. by foaming, bloating or extruding through a channel, orifice and/or nozzle to obtain the low density of the liquid collagen in the specified range.

This provides for a viscous, but light and fluffy preparation that readily mixes with blood at a hemorrhaging site and is intimately contacting and adhering to the wound surface. It is required that the hemostat according to the invention contains the collagen as a liquid or gel, thus avoiding fibrillar undissolved collagen which could form a barrier of contact and reaction with the blood. Thereby the relatively good resorption of the hemostat by the body is a specific advantage of the product according to the invention, which aims at the quick hemostasis and also at avoiding complications by undesired adherences in the body. The resorption time of the hemostat according to the invention usually is lower than 4 weeks, mostly less than 3 weeks. In some cases the hemostat may even be no more visible 1 week after its application.

Preferred preparations according to the invention have a translucent or transparent look resulting from the collagen that is in a gel state and does not contain particulate solids dispersed therein. Collagen preparations based on native or atelopeptide collagen specifically make up a glass-like appearance. The hemostat according to the invention may nevertheless be solidified to obtain a presentation form that is similar to a sponge, film or sheet. Although it is solidified it still has the collagen gel properties which are evident after reconstitution to the flowable gel. The solidification may be obtained after drying to a water content of 1-25%, preferably between 10 and 25% water (w/w). The drying can simply be effected by floating the collagen liquid on plates, in blisters or in adequate forms and drying with air or a drying gas whereby the foamy structure and the low specific weight of the collagen hemostat is preserved.

After drying the solidified liquid can be cut or shaped as required, just like any solid hemostat. Exemplary presentation forms are pads, patches, cubes, tubes, cones and granules.

Since the solidified gel is still a glassy liquid it maintains its elastic properties and absorption capacities related to the large pores that resulted from the expansion process. The advantage of the solidified gel is the improved storage stability. It is thus possible to provide a storage-stable solidified liquid hemostat that can be stored at room temperatures up to 35° C. If thermolabile drugs are incorporated in the hemostat, it may however be necessary to keep the storage temperature in the range of 2-8° C.

The solidified gel is easily reconstituted to the original liquid state either before its application by addition of water or physiological solutions, optionally containing drugs, or directly at the wound site by contact with wound fluid and blood.

The liquid collagen is storage stable as well. In some instances depending on the water content, the origin and type of collagen, it can be kept at temperatures up to 35° C., preferably at room temperature (20-25° C.). However, if it further comprises thermolabile drugs, it should be stored at refrigerating temperatures (2-8° C.), or else a stabilizer admixed to the collagen and/or the drug. The storage of the hemostat or the liquid collagen as a frozen preparation, preferably at temperatures of −30 to −10° C. may be a favorite option for providing storage stability of the product, which is at least 2 years.

It is further preferred to store the hemostat as the liquid collagen before expansion, for instance, if the expansion of the collagen is just before application of the hemostat, e.g. by pressure through an orifice or extrusion channel. This procedure ensures the low density of the freshly prepared hemostat, in particular when a gas is introduced immediately. Therefore it is adequate to use an aerosol container to obtain a foam upon release of the product through the orifice of the container.

According to a preferred embodiment the hemostat according to the invention contains one or more specific additives facilitating the foaming or extruding process. These additives are foaming additives, in particular selected from the group consisting of tensides, carbohydrates and salts of organic acids.

The preferred tensides are non-ionic, anionic or cationic tensides, such as organic polymers, polyglycols or quarternary ammonium salts, in particular benzalkonium chloride. The preferred concentration is in the range of 0.01 to 10 g per liter, more preferred 0.1 to 5 g per liter, in particular 1 to 2 g per liter.

Also carbohydrates like sugars or sugar alcohols, specifically glucose or saccharose, have favorable actions on the foaming capability of the hemostat according to the invention. The carbohydrate concentration preferably is in the range of 1 to 10 g per liter, more preferred 2 to 8 g per liter, mostly a concentration of 3 to 5 g per liter is used.

Exemplary salts of organic acids that may be used as foaming agents are calcium or alkali salts of acetic, lactic or citric acid, or generally soluble salts of low molecular organic salts. The preferred concentration in this case is 0.5 to 10 g per liter, more preferred 1 to 5 g per liter, usually 2 to 3 g per liter.

The most preferred mixture of foaming agents are benzalkonium chloride, glucose or saccharose and calcium lactate.

On the other hand a foaming agent such as a stabilizer may be used as an additive to keep the low density structure of the liquid hemostat for a prolonged period of time, usually at least 2 hours, preferably at least 12 hours, in some cases up to 48 hours. It is specifically useful to choose the type and concentration of stabilizer to ensure the foamy constituency for the period of 1 to 8 hours after expanding the liquid collagen, to provide a ready to use hemostat even during a long term surgical intervention.

The storage of the hemostat before its expansion may well be feasible in a container for immediate application of the expanded hemostat, ready to use. The container of choice has preferably means for introducing a propellent or a gas, such as a can or aerosol container that already contains a pressurized gas, a hydrofluorocarbon gas or the like. It is further preferred to provide the hemostat according to the invention in a container that is equipped with an extrusion channel, an orifice or a nozzle as a means for expanding the liquid collagen. The hemostat can be applied through the specific expansion means of the container by pressure on the container using a piston or by manual pressure on a flexible container, such as a flexible tube. A preferred container may be a syringe equipped with an appropriate cannula as extrusion channel.

The further subject of the invention is a method for preparing the collagen hemostat starting from a source containing native collagen, acidifying the material, heating the collagen under agitation, neutralizing and expanding the collagen to the low density hemostat according to the invention.

The source material for producing the hemostat according to the invention is preferably collagen from mammalian sources, most preferred from species which bear reportedly no risk of transmissible spongiform encephalitis (TSE). The pathogens of TSE are not yet characterized. It is however known that cows, sheep and goats may contain pathogens causing transmissing spongiform encephalitis, like BSE (bovine spongiform encephalitis), scrapie (sheep TSE) or CJD (Creutzfeld Jakob Disease). According to the recommendation of the world health organization WHO the source of collagen material for use in pharmaceuticals should be carefully selected to reduce the risk of transmitting those pathogens, also known as prions. The further recommendation is a treatment with chemicals to reduce the potential infectivity of the product. A preferred method is the treatment of collagen with a high concentration of NaOH. The treatment with the chemicals further reduces the risk of transmitting other pathogens like viruses that could be harmful to humans.

Collagen from horses, pigs or birds, even from humans are preferred sources for the hemostat according to the invention. However, provided the security recommendations of the WHO are considered also bovine, or other ruminant sources may be used. The material may be derived from tendons, skins or placenta. It is preferred to use native collagen that is extracted using a procedure to maintain the integrity of the polypeptide chains and the helical structure of the molecule.

It may also be treated with enzymes such as pepsin to obtain atelopeptide collagen, which specifically results in the collagen hemostat of the translucent or transparent look. Native, including atelopeptide collagen specifically comprises all fractions of collagen including the acid soluble and acid insoluble fraction. Such a native material has significantly improved hemostatic activity.

Yet it is not preferred to use collagen modified by chemical reactions, like cross-linking. Different collagen types, like type I, II, III or IV can be treated to produce the hemostat according to the invention, the preferred collagen is however of type I or a preparation containing more than 80% type I collagen.

The collagen is contained in the source material mostly in the fibrillar form. The fibrils are then dissolved to form a collagen liquid in an acid milieu, preferably at a pH between 3 and 5. The collagen is advantageously acidified by addition of acetic acid, citric acid, lactic acid or monochlor acetic acid. It was surprising that the specific heating of the collagen liquid to a temperature of 30 to 50° C. under agitation before or after neutralization using alkali solutions, is found to enable the expansion of the liquid to form the low density hemostat according to the invention. In the case of a low, denaturation temperature of the collagen it may be required to control the temperature not to exceed a temperature of 40° C., preferably to achieve a temperature between 30 and 38° C. Agitation by directly stirring the liquid is possible. The heating is preferably employed during a time period of 5 min to 2 hours, usually between 10 and 90 minutes, in a container using a stirrer to employ 100-10000 tours per minute, usually between 500 and 7000 tours per minute, preferably between 1000 and 5000 tours per minute. The intensity of heating and agitation mainly depends on the viscosity of the product, the origin and type of collagen.

The neutralization is usually effected to a pH between 6 and 8, preferably between 6.5 and 8.0, at low temperatures, like below 30° C.

The method according to the invention may further comprise the process steps of washing or cutting the collagen source material, delipidation and optionally elimination of aminoglycans by treatment with salts.

The liquid collagen further may be characterized by a relatively high viscosity between 20 and 10000 cp, preferably between 50 and 5000 cp. Nevertheless the product is easily applied to the wound site because of its low density in the extended form.

The hemostat according to the invention is further provided as a sterile material. The process of manufacturing the hemostat or intermediate materials thus comprises the process step of sterilization. It is hereby advantageous to either sterilize the collagen by chemical treatment, filtration through a bacteria filter or by irradiation using beta or gamma rays, for example with an intensity of 15-30 kGy, usually about 25 kGy. The collagen may be chemically treated using ethylene oxide or peroxides.

According to a specific embodiment of the invention the collagen hemostat further comprises physiologically active substances or drugs, preferably in the dissolved form or as a dispersion. Among the optional additives are antibiotics, antimycotics, antiviral substances, antiphlogistics, analgesics and factors of the blood coagulation and fibrinolysis system. Antibiotics, like gentamycine, may be preferred when the local application of the substance should challenge septic conditions.

The addition of a human factor of blood coagulation, like a blood coagulation factor selected from the group consisting of factors I, II, V, VII, VIII, IX, X, XI, XIII, von Willebrand Factor, Fibronectin, Vitronectin, Protein C and Protein S, may positively influence the kinetics of hemostasis by the reaction with the intrinsic and extrinsic system of coagulation to support secondary hemostasis.

Further platelet factors, enzymes or inhibitors of coagulation and fibrinolysis may be included. The most immediate action may be obtained by using activated factors of coagulation to provoke a quick secondary hemostasis. It is for instance preferred to include thrombin in the hemostat according to the invention to get a synergistic action of primary and secondary hemostasis at the bleeding wound. The thrombin can be stabilized using proteins like human serum albumin, amino acids, polyglycols or other carbohydrates, like sugars and sugar alcohols, for a storage stable liquid at refrigerating or ambient temperatures. When using a frozen thrombin preparation the stabilizer concentration may be reduced to a minimum, and even be spared.

The preferred thrombin concentration in the collagen hemostat according to the invention is in the range of 1 to 70 IU/ml, more preferably between 5 and 60 IU/ml.

Further possible additives are the above mentioned foaming additives, wound healing promoting agents, growth factors, glucocorticosteroids, steroids, vitamins or vitamin derivatives, tumoricidal or tumoristatic compounds, minerals, immunomodulators, immunoglobulins, dyes, radiolabels, fluorescent labels, polysaccharides, anesthetics and nucleic acids.

When using biological additives derived from human sources care should be taken not to transmit human pathogenic agents, such as blood born viruses. Among the relevant viruses the HIV, Hepatitis Viruses and Parvoviruses are well-known. Therefore effective methods of inactivating and/or depleting potentially present viruses in the preparation of the biologicals or additives are employed. Those methods are for instance the treatment with solvent and detergent, such as according to the EP 0 131 740, the heat treatment methods of pasteurization or heating in the dry state, irradiation methods and filtration methods.

According to a preferred manufacturing method the additives are directly mixed to the collagen before it expansion. However, another preferred option is to include the above mentioned additives, such as thrombin, to the hemostat according to the invention during the expansion of the collagen using a separate container for the solid or dissolved additive to be extruded by applying pressure. Thus a two chamber container destined to contain both collagen liquid and the additive as separate components and means to mix the components before application or at the site of application is preferred.

The further possible addition of one or more of the above mentioned physiologically active substances or drugs is the direct coating of the solidified liquid hemostat according to the invention with the additive. A coating layer containing the additive and optionally a carrier substance, like a bioresorbable polymer, preferably collagen, is applied to the solidified liquid after drying the hemostat.

This may be effected by overlaying the hemostat with a solution or dispersion of the additive and optionally the carrier mixture in a solute, and evaporating the solute to leave the additive and optionally the carrier substances as a coating on the surface. As a suitable solute water or an organic solute like ethanol may be used.

For most surgical procedures it is required to achieve hemostasis within 3 to 5 minutes. The collagen hemostat according to the invention fulfils this requirement, and occasionally stops bleeding within 1 or 2 minutes.

Since the hemostat according to the invention contains a relatively high concentration of collagen in the liquid and however has low density due to the expansion process, it is very well suitable for punctual application during surgical interventions. It may however also be applied to large hemorrhaging wounds, and most suitably in cases of diffuse and oozing bleeding.

It is also very useful for being placed in contact with any hemorrhagic site of uneven surface and more particularly in thoracic, orthopedic, plastic (skin grafts), ENT or neurosurgery. It may as well be applied at a puncture site of organ tissue. A preferred mode of application is by laparoscopic procedures. The indication of microinvasive or minimal invasive surgery is most interesting for such a flowable and easily applicable material like the hemostat according to the invention, especially in the abdomen, the thoracic and cardiovascular indications. Generally, hemostasis at all wound sites may be achieved. This includes surgical wounds, burns, ulcers and lacerations.

The following example is illustrating the present invention.

### EXAMPLE 1

### Preparation of the Collagen Hemostat

150 g dry collagen powder obtained by extraction from horse skin and sterilized by irradiation are dissolved in 10 liters of acetic acid solution to get a pH of 4. It is then neutralized by the addition of sodium hydroxide to a pH of 7.

The mixture is first agitated for 10 minutes at 500 tours/min and room temperature. Saccharose is added to a concentration of 0.5% (w/w). Following the agitation process of 3 to 4 minutes the further additive of calcium lactate is mixed to the collagen to a concentration of 0.2% (w/w). The next agitation period is 5 minutes at 1000 tours per minute. After addition of benzalkonium chloride to a concentration of 0.2% the mixture is agitated for 15 minutes at 1000 tours per minute, and the temperature increased to 40° C.

The collagen liquid is then filled in a syringe and extruded through an adequate cannula by manually pressing to obtain a foamy hemostat. The density of the hemostat was 0.6 mg/cm³.

1. Collagen hemostat based on liquid collagen of pH 6-8 that is expanded to a specific weight in the range of 0.07-0.9 mg/cm³.

2. Hemostat according to claim 1, characterized in that the collagen concentration in the liquid collagen is in the range of 1-40 mg/ml, preferably 1-20 mg/ml.

3. Hemostat according to claim 1 or 2, characterized in that it contains a foaming additive selected from the group consisting of tensides, carbohydrates and salts of organic acids.

4. Hemostat according to claim 1 to 3, characterized in that it has a translucent or transparent look.

5. Hemostat according to claim 1 to 4, characterized in that it is a solidified liquid.

6. Hemostat according to claim 5, characterized in that it is air-dried.

7. Hemostat according to claim 5 or 6, characterized that it has a water content of 1-25%, preferably 10-25%.

8. Hemostat according to claim 1 to 6, characterized in that it is combined with a physiologically active substance.

9. Hemostat according to claim 8, characterized in that the physiologically active substance is selected from the group consisting of antibiotics, antimycotics, antiviral substances, antiphlogistics, analgesics and factors of the blood coagulation and fibrinolysis system.

10. Hemostat according to claim 9, characterized in that the physiologically active substance is a blood coagulation factor, optionally in its activated form.

11. Hemostat according to claim 8-10, characterized in that it is combined with the physiologically active substance as a solution.

12. Hemostat according to claim 8-10, characterized in that it is combined with physiologically active substance as the solidified liquid according to claim 5-7.

13. Hemostat according to claim 12, characterized in that it contains the physiologically active substance in a coating layer.

14. Hemostat according to claim 13, characterized in that the coating layer comprises a mixture of collagen and the physiologically active substance.

15. Hemostat according to claim 1-14, characterized in that is provided in a pressurized container.

16. Hemostat according to claims 1-15, characterized in that it is provided in a container with means for introducing a propellent.

17. Method of preparing a collagen hemostat according to claim 1 to 16 by acidifying a source of native collagen, heating the collagen to a temperature of 30-50° C. under agitation, neutralizing the collagen by addition of an alkaline solution and expanding the collagen to obtain a liquid of low density.

18. Method according to claim 17, characterized in that the collagen is expanded by extrusion through an orifice and/or a nozzle by pressure on the liquid, optionally using a propellent.

19. Method according to claim 17 or 18, characterized in that a foaming additive is added before expanding the collagen.

20. Method according to claim 19, characterized that the liquid is dried to a water content of 1-25%, preferably to 10-25%.

21. Method according to claim 17-20, characterized in that it comprises the addition of a physiologically active substance, such as a blood coagulation factor, optionally in its activated form.

22. Method according to claim 21, characterized in that the physiologically active substance is added before or during expanding the collagen.

23. Method according to claim 21, characterized in that the physiologically active substance is added by a coating layer.

24. Method for preparing a collagen hemostat according to any of claims 1 to 16, for use in surgical interventions.

25. Method according to claim 24, for use in minimal invasive surgery.
